(19) United States
(12) Patent Application Publication
(10) Pub. No.: US 2006/0036323 A1
(43) Pub. Date: Feb. 16, 2006

(54) Facet device and method

(76) Inventors: Alan L. Carl, Slingerlands, NY (US); Dan Sachs, Minneapolis, MN (US); Meir Rosenberg, Newton, MA (US)

Correspondence Address: Susan Schmitt;PETERS, VERNY, JONES & SCHMITT LLP, Suite 230, 425 Sherman Avenue, Palo Alto, CA, 94306, US

(21) Appl. No.: 11/197,566

(22) Filed: Aug. 3, 2005

Related U.S. Application Data
(60) Provisional application No. 60/598,882, filed on Aug. 3, 2004.

(51) Int. Cl. A61F 2/44

(52) U.S. Cl. 623/17.11

(57) ABSTRACT

A spine prosthesis is provided and in particular, related to the facet joint of a spine.

### RELATED APPLICATION DATA

The present application claims the priority of Provisional Application No. 60/598,882 filed Aug. 3, 2004 and entitled: Spine Treatment Devices and Methods.

### FIELD OF THE INVENTION

The invention relates to devices to treat the spine, in particular in association with a facet joint, including but not limited to spinal stabilization devices, spinal distraction devices, spinal prostheses, devices to treat pain associated with the spine, and other spinal treatment devices.

### GENERAL BACKGROUND

Certain spine conditions, defects, deformities (e.g., scoliosis) as well as injuries may lead to structural instabilities, nerve or spinal cord damage, pain or other manifestations. Back pain (e.g., pain associated with the spinal column or mechanical back pain) may be caused by structural defects, by injuries or over the course of time from the aging process. For example, back pain is frequently caused by repetitive and/or high stress loads on or increased motion around certain boney or soft tissue structures. The natural course of aging leads to degeneration of the disc, loss of disc height, and instability of the spine among other structural manifestations at or around the spine. With disc degeneration, the posterior elements of the spine bear increased loads with disc height loss, and subsequently attempt to compensate with the formation of osteophytes and thickening of various stabilizing spinal ligaments. The facet joints may develop pain due to arthritic changes caused by increased loads. Furthermore, osteophytes in the neural foramina and thickening of spinal ligaments can lead to spinal stenosis, or impingement of nerve roots in the spinal canal or neural foramina. Scoliosis also creates disproportionate loading on various elements of the spine and may require correction, stabilization or fusion.

Pain caused by abnormal motion of the spine has long been treated by fixation of the motion segment. Spinal fusion is one way of stabilizing the spine to reduce pain. In general, it is believed that anterior interbody or posterior fusion prevents movement between one or more joints where pain is occurring from irritating motion. Fusion typically involves removal of the native disc, packing bone graft material into the resulting intervertebral space, and anterior stabilization, e.g., with intervertebral fusion cages or posterior stabilization, e.g., supporting the spinal column with internal fixation devices such as rods and screws. Internal fixation is typically an adjunct to attain intervertebral fusion. Many types of spine implants are available for performing spinal fixation, including the Harrington hook and rod, pedicle screws and rods, interbody fusion cages, and sublaminar wires.

Alternatives have been proposed and tested to replace the need for spinal fusion to treat patients with back pain. These implants include artificial discs and artificial nucleus technologies that preserve motion. However, these implants do not directly address the forces borne by the facet joints.

The facet joints provide a means for load transmission, support and motion of the posterior spinal column. Disc height loss from degenerative disc disease and aging leads to increased load on the facet joints, which can lead to arthritic, painful, degenerative changes.

Often over the course of degenerative disc disease there is a narrowing of the neural foramen through which the nerves exit the spine. In addition to the degeneration of discs causing the narrowing of the foramen, there is also calcification around the foramen causing further narrowing or stenosis resulting in pain to the patient. Currently, these conditions may be treated by removing some or all of the lamina (laminectomy) or posterior bone adjacent or around the stenotic neural foramen

Given that the facet joint and its environs is a source of pain for some patients, some procedures have been developed or proposed to relieve pain associated with the facet joint. Partial or complete removal of the pathological facets, and replacement with a mechanical joint that preserves motion similar to a facet has been proposed. Additionally, individual degenerative facet articulations have been replaced with caps.

It would be desirable to provide improved devices and methods for relieving pain associated with the facet joints.

Spinal stenosis pain or from impingement of nerve roots in the neural foramina has been treated by laminectomy and foraminotomy, and sometimes reinforced with rod and screw fixation of the posterior spine.

More recently, as an alternative to laminectomies and related procedures, implants have been proposed that distract the spine from a posterior approach. In particular, a wedge-like implant inserted between two adjacent spinous processes has been proposed to relieve pressure on spinal nerves and nerve roots. A kyphosis is induced, which opens the space of the spinal canal and neural foramen, thereby reducing the effect of spinal stenosis. However, this type of distraction of adjacent spinous processes is suboptimal for several reasons: The resulting kyphosis is non-physiologic, leading to increased load on the anterior portion of the disc and the vertebral bodies. This can increase the risk of disc degeneration and vertebral compression fracture. The implant tends to bend the spine forward. The spinous processes may fracture due to the distraction forces of the wedge implant. Bone may collapse around the spinous process. The implant may weaken, tear, or stretch stabilizing ligaments of the spine, such as the supraspinous ligament, interspinous ligament, ligamentum flavum, posterior longitudinal ligament, or capsule of the zygapophyseal joint. The amount of distraction is not adjustable to the specific amount of stenosis, and cannot be easily readjusted months to years after the device has been implanted.

It would accordingly be desirable to provide a distraction device that reduces or avoids some or all of these issues.

Pain due to instability of the spine has also been treated with dynamic stabilization of the posterior spine, using elastic bands that connect pedicles of adjacent vertebrae.

The typical techniques for fusion, decompression, and dynamic stabilization require open surgical procedures with removal of stabilizing muscles from the spinal column, leading to pain, blood loss, and prolonged recovery periods after surgery due in part to the disruption of associated body structures or tissue during the procedures.

Accordingly, it would be desirable to provide less invasive devices and methods for treating pain or discomfort associated with the spinal column. It would also be desirable to provide such devices and methods that are less damaging to associated tissue.

Spine surgeons commonly use metallic or polymeric implants to effect or augment the biomechanics of the spine. The implants frequently are attached or anchored to bone of the spine. Sites typically considered appropriate for boney attachment have high density or surface area, such as, for example, the pedicle bone, the vertebral body or the cortical bone of the lamina. The spinous process contains thin walls of cortical bone, and thus, has been considered as not ideal for anchoring spinal implants as they may not support the implants under physiologic loads, or the intermittent high loads seen in traumatic situations. Fixation has been attempted from spinous process to spinous process with poor results.

A translaminar facet screw as used by some surgeons goes through the base of spinous process to access the cancellous bone of the lamina. A disadvantage of this device is that it is not suitable for attaching to a pedicle screw and the depth and angle during deployment can be very difficult to track or visualize, thus increasing the possibility that the screw would extend into the spinal canal. A facet screw is screwed between opposing facets of a zygapophyseal joint.

### SUMMARY

One aspect of the present invention is directed to providing a device and method for alleviating discomfort and or deformity associated with the spinal column. Another aspect of the present invention is directed to providing a minimally invasive implant and method for alleviating discomfort associated with the spinal column. Another aspect of the present invention provides an anchoring device and method that requires less surrounding tissue damage or disruption. Another aspect of the present invention provides reinforcement of the spinous process for use in various spinal systems. Another aspect of the invention provides a minimally invasive, non-invasive, or remote adjustment or lengthening of an orthopedic device. Another aspect of the invention provides a minimally invasive, non-invasive, or remote adjustment, lengthening or shortening of a stabilization device. Another aspect of the present invention also provides an implant system and device suitable for minimally invasive, minimally disruptive and/or percutaneous posterior deployment across a plurality of motion segments and more than two motion segments. Different aspects of the invention may provide distraction forces to relieve pressure on certain structures, compression forces to fix (“fix” as set for the herein shall mean to fix either directly or indirectly and may include dynamic elements) or stabilize motion across structures, shock absorbing qualities to help relieve load from certain structures, and therapeutic activity to reduce inflammation and pain. Other aspects of the invention may supplement or bear load for degenerated, painful, or surgically removed joints, e.g., the facet joint. Another aspect of the invention may provide a method and system for treating deformities such as scoliosis. Other aspects of the invention may include sensors associated with implants or implanted at or near the bones, soft tissue, or joints of the spine and may provide feedback regarding the joint on an ongoing basis. The sensors may also be part of a feedback system that alters a property of an implant in response to sensing information. Another aspect of the invention may provide a device or method for delivering therapeutic substances at or near the spine.

One aspect of the invention provides for repair or reconstruction of a dysfunctional facet joint. For example, by entering the capsule of the facet joint, creating a space between articulating facets by removing synovium, cartilage, and some bone from within the zygapophysial joint, and, then, inserting a motion preserving prosthesis. Motion preserving prostheses may include a smooth and/or curved surface, a sphere, an egg shaped/oval implant, or a self contained “ball and socket” joint. Magnetic plates with like poles facing each other may be attached to interfacing articulating portions of the facets. Attachment of the motion preserving prosthesis may involve extensions from the prosthesis that partially or completely penetrate each of the facets.

Another aspect of the invention provides for repairing the encapsulating ligaments with suture, adhesive, a patch, or other materials after a capsule of the zygapophysial joint has been invaded for tissue removal and insertion of a prosthesis. One aspect of the invention includes an elastic encapsulating wrap used to stabilize the facet joints. Another aspect of the invention provides a stabilizing or distraction rod used to keep each facet in apposition, thereby keeping the prosthesis in place. In accordance with an aspect of the invention, the stabilizing or distraction rod may be placed between ipsilateral pedicles of each articulating segment, between contralateral pedicles, between the spinous process and pedicle, or between the lamina and pedicle.

According to an embodiment of the invention, a facet distraction implant is provided for maintaining a space that is formed between the facet articulations of adjacent vertebrae when the joints are distracted. The facets may be distracted using a known distraction method or technique and an implant may be placed between the facets. A securing device according to the invention may be positioned to anchor each of the facet articulations of a facet joint to each other in distraction to maintain the opening of the corresponding neural foramen. The prosthesis may include a distraction element that exerts a distracting force on the joint.

According to another aspect of the invention, a facet joint replacement or augmentation may comprise a stabilizing prosthesis placed through a spinous process of a first vertebra associated with the facet joint to be replaced, across or adjacent the location of the removed or partially removed facet and anchored in a bony portion of an adjacent second vertebra associated with the facet joint to be replaced, i.e. pedicle, transverse process, lamina or other bony portion. The stabilizing prosthesis may include a dynamic portion that permits some movement of the stabilizing device. The stabilizing device may also be bilateral.

According to another embodiment, the facet replacement stabilizing device may be anchored to contralateral pedicles of adjacent vertebrae. The stabilizing device may also be bilateral.

According to another aspect of the invention a facet joint replacement or augmentation may comprise a distracting prosthesis placed through a spinous process of a first vertebra associated with the facet joint to be replaced or across or adjacent the location of the removed or partially removed facet and anchored in a bony portion of an adjacent second vertebra associated with the facet joint to be replaced. The distracting prosthesis may include a dynamic portion that permits some movement of the stabilizing device. The distracting device may be bilateral.

In another embodiment, a distracting device may be anchored to contralateral pedicles of adjacent vertebrae. The distracting device may also be bilateral.

In accordance with one aspect of the invention, a reinforcement structure is provided for supporting the spinous process and if desired, in addition, the lamina of a spine. The invention further provides a method and system for forming or implanting such structure in the spinous process or a region of cancellous bone in the lamina of a spine. The reinforcement system may include one or more systems of reinforcement and may be used before, during and/or after a spinal device (e.g. a stabilization, distraction or prosthetic device, etc.) is coupled to the spinous process.

Various aspects of the invention are set forth in the description and/or claims herein.

### BRIEF DESCRIPTION OF THE DRAWINGS

FIG. 1 is a schematic side view of a facet implant in accordance with the invention.

FIG. 2 is a schematic side view of a facet implant in accordance with the invention.

FIG. 3 is a schematic posterior lateral perspective view of a facet implant in accordance with the invention.

FIG. 4 is a side partial cross section of a facet implant in accordance with the invention.

FIG. 5 is a side partial cross section of a facet implant in accordance with the invention.

FIG. 6 is a schematic posterior lateral perspective view of a stenotic neural foramen of a posterior spine.

FIG. 7 is a schematic posterior lateral view of a facet implant in accordance with the invention.

FIG. 8 is a side schematic view of a facet implant in accordance with the invention.

FIG. 9 is a side schematic view of a facet implant in accordance with the invention.

FIG. 10 is a side schematic view of a facet implant in accordance with the invention.

FIG. 11 is a side schematic view of a facet implant in accordance with the invention.

FIG. 12 is a side schematic view of a facet implant in accordance with the invention.

FIG. 13 is a side schematic view of a facet implant in accordance with the invention.

FIG. 14A is a posterior lateral perspective view of an implant adjacent a removed joint segment in accordance with the invention.

FIG. 14B is a posterior view of the implant implanted as shown in FIG. 14A.

FIG. 15 is a schematic side view of a connector of an implant in accordance with the invention.

FIG. 16 is a schematic side view of a connector of an implant in accordance with the invention.

FIG. 17 is a schematic perspective view of a connector in accordance with the invention.

FIG. 18A is a side schematic view of a distraction element in a first position in accordance with the invention.

FIG. 18B is a side schematic view of the distraction element of FIG. 18A in a second position in accordance with the invention.

FIG. 18C is a side schematic view of a distraction element in a first position in accordance with the invention.

FIG. 18D is a side schematic view of the distraction element of FIG. 18C in a second position in accordance with the invention.

FIG. 18E is a side schematic view of a distraction element in accordance with the invention.

FIG. 18F is a side schematic view of a distraction element in accordance with the invention.

FIG. 19 is a schematic side perspective view of a dynamic element in accordance with the invention.

FIG. 20 is a schematic side perspective view of an adjustable implant element in accordance with the invention.

FIG. 21 is a schematic side perspective view of an adjustable implant element in accordance with the invention.

FIG. 22 is a schematic side perspective view of an adjustable implant element in accordance with the invention.

FIG. 23A is a lateral posterior view of a vertebra with a reinforcement structure in accordance with the invention.

FIG. 23B is a side view of the vertebra and reinforcement structure of FIG. 23A.

FIG. 24A is a lateral posterior view of a vertebra with a reinforcement structure in accordance with the invention.

FIG. 24B is a side view of the vertebra and reinforcement structure of FIG. 24A.

FIG. 25A is a lateral posterior view of a vertebra with a reinforcement structure in accordance with the invention.

FIG. 25B is a side view of the vertebra and reinforcement structure of FIG. 25A.

FIG. 26A is a lateral posterior view of vertebrae with a reinforcement structure and implant in accordance with the invention.

FIG. 26B is a side view of the reinforcement structure and implant of FIG. 26A.

FIG. 26C is a top view of a reinforcement structure and implant in accordance with the invention.

FIG. 26D is a posterior view of the reinforcement structure and implant of FIG. 26C.

FIG. 27 is a posterior view of a reinforcement structure and implant in accordance with the invention.

FIG. 28 is a posterior view of a reinforcement structure and implant in accordance with the invention

FIG. 29 is a schematic view of an adjustable pedicle attachment device in a first position in accordance with the invention.

FIG. 30 is a schematic view of the adjustable pedicle attachment device of FIG. 29 accordance with the invention.

FIG. 31 is a schematic posterior lateral perspective view of a therapeutic substance delivery device in accordance with the invention.

FIG. 32 is a schematic posterior lateral perspective view of a therapeutic substance delivery device in accordance with the invention.

### DETAILED DESCRIPTION

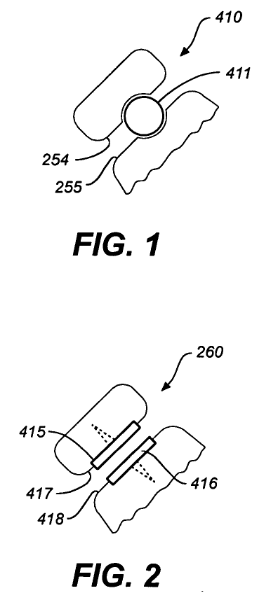

FIGS. 1-5 illustrate facet repair prostheses in accordance with an embodiment of the invention. Prosthesis 410 comprises a ball bearing 411 implanted between the caudal and the cephalic facets 412, 413 of the zygapopyhseal joint 414. (FIG. 1) The joint 414 is prepared by removing soft tissue between the joints and creating a concavity on adjacent facet plates for receiving the ball bearing.

In FIG. 2, magnets 415, 416 including smooth interacting bearing surfaces are respectively screwed into the cephalic and caudal facets 417, 418 of the zygapopyhseal joint 419. The magnets 415, 416 are oriented so that like poles face each other (e.g. North-North or South-South) to provide a distraction force at the joint. The magnets may have a center hole through which a rod is inserted to resist the tendency of one magnet to move relative to the other. Each end of the rod may have a diameter larger than the center holes. This system may be used in other joints in the body to maintain separation between the joints.

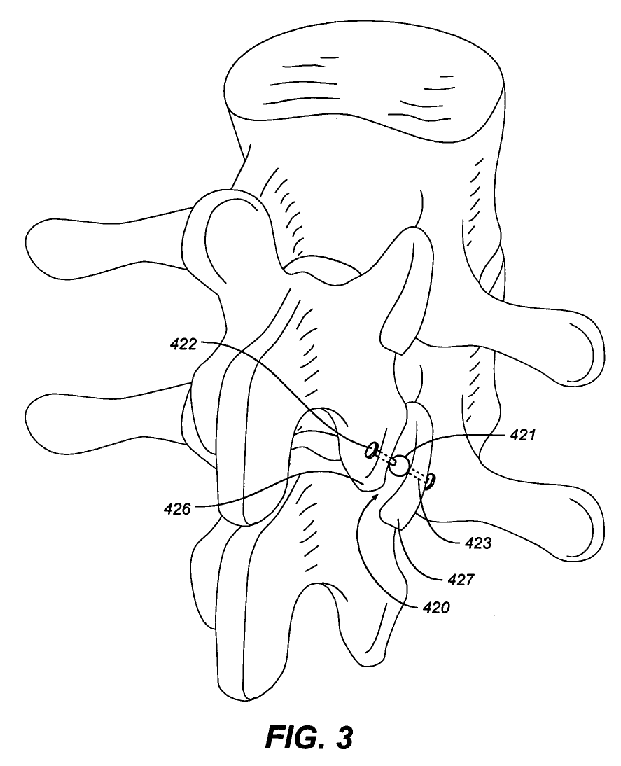

Referring to FIG. 3, a joint prosthesis 420 is positioned between the cephalic and caudal facets 426, 427. The prosthesis comprises a ball 421 providing a bearing surface for the motion of the facets 426,427, and opposing posts 422, 423 screwed in or otherwise implanted in the facets 426, 427, respectively for securing the ball 421 within the joint 428. The ball 421 may include openings for receiving the posts, e.g., in a tapered interference type fitting, to secure the posts 422, 423 to the ball 421 and to secure the ball 421 within the joint 428.

This facet repair may be performed percutaneously or via minimally invasive surgical techniques, for example using percutaneously positioned distracting instruments to distract the joint, for example, an expanding balloon or forceps like distractors. Using a hollow needle percutaneously positioned into the joint, an expandable or self-expanding facet distraction implant may be placed in position through the hollow lumen of the needle into the joint. A polymer material may be injected into the joint through a percutaneously inserted needle.

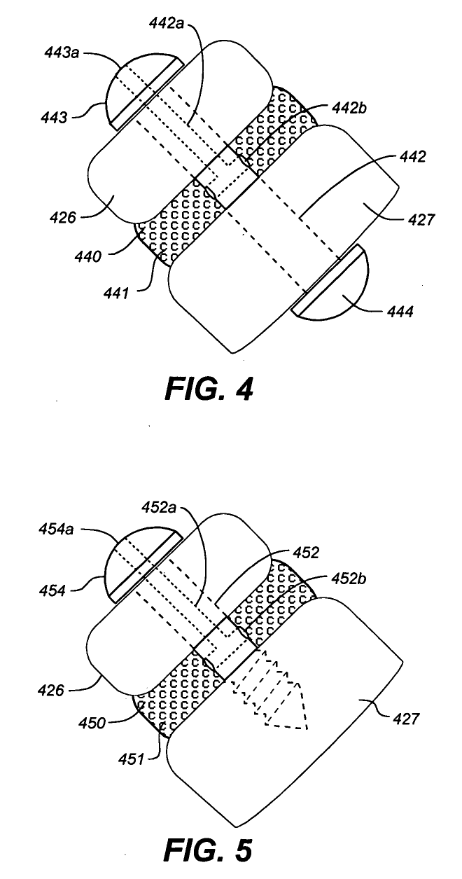

FIG. 4 illustrates a material 440 such as a polymer injected between the cephalic and caudal facets 426, 427. The material 440 forms a flexible member 441 that allows some movement of the joint due to the flexible properties and/or the shape that permit articulation of the joint. A securing member 442 extends through the facets 426, 427 and the material 440 to further hold the member 441 in place in the joint capsule and/or to prevent implant extrusion. The securing member 442 includes anchors 443, 444 that anchor to the outside or within the facets 426, 427 to hold the securing member 442 in place while permitting some motion for example through spacing at or in the joint. The securing member 442 may for example, comprise a screw, or may be constructed of a flexible material such as a flexible polymer. The securing member may also comprise a band constructed of fibers strands such as Kevlar™, polypropylene or polyethylene, or constructed of a fiber reinforced polymer. The anchors 443, 444 may be of a material such as titanium, or PEAK that may be screwed or crimped on to the securing member 442. The polymer may be injected into the joint capsule into opening 443a in the anchor 443, through a lumen 442a in the securing member 442 and through holes 442b or pores in the securing member 442. This may be done when the joint is distracted or otherwise positioned as desired.

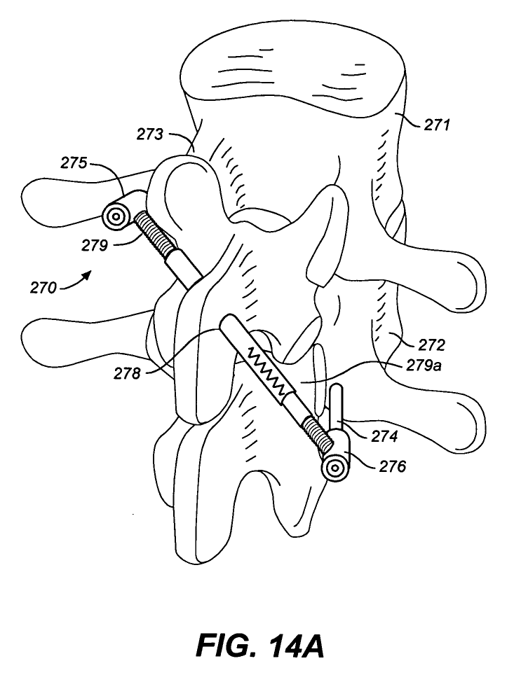

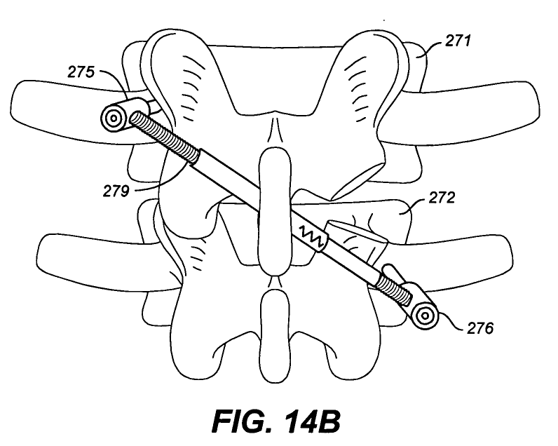

FIG. 5 illustrates a material 450 such as a polymer injected between the cephalic and caudal facets 426, 427. The material 450 forms an implant 451 that allows some movement of the joint due to the flexible properties and/or a shape that permits articulation of the joint. A securing member 452 extends through the facets 426, 427 and the material 450 to further hold the implant 451 in place in the joint capsule. The securing member 452 includes anchor 453 that anchors the member to the outside or within the facet 426, (or alternatively to the outside or within the facet 427) to hold the securing member 452 in place. The securing member further 452 includes tapered end that allows the securing member 452 to be inserted through the joint capsule and anchored into facet 427. The securing member may be a screw with threaded tip 454 that screws into the bone. The threaded tip may be fixed to the flexible portion 455 that may be constructed in a similar manner as securing member 442 described with reference to FIG. 14B. The securing member 452 further includes a flexible portion 455 that allows some movement of the securing member 452 and joint. The anchor 453 may include an opening 453a into a lumen 452a in the securing member 452, for injecting a polymer into a lumen 452a in the member and then through holes 452b into the joint capsule to form the implant 451.

According to the invention, a facet joint device as described herein may be used in combination with an artificial disc or other spinal implants, e.g., to maintain the integrity of the facets. The facet joint distraction or replacement devices and procedures described herein may be used in conjunction with anteriorly placed implants, e.g., in a load sharing arrangement. The facet joint resurfacing, distraction or augmentation as well as the anterior implants may be used with a process to pedicle distraction or stabilizing device as described herein. Various spinal implants may also be used with facet resurfacing, facet distraction or augmentation procedures.

In accordance with one aspect of the invention, narrowing or stenosis of the neural foramen may be treated using a device configured to distract the facet joint. Accordingly, a distraction system is provided for distracting the facet joint.

Referring to FIG. 6, a portion of the spine is illustrated with adjoining vertebrae prior to distraction. The neural foramen 250 between a first vertebra 251 and a second vertebra 252 is stenotic. At the zygapophyseal joint capsule 253, there is no gap between the cephalic and caudal facets 254, 255.

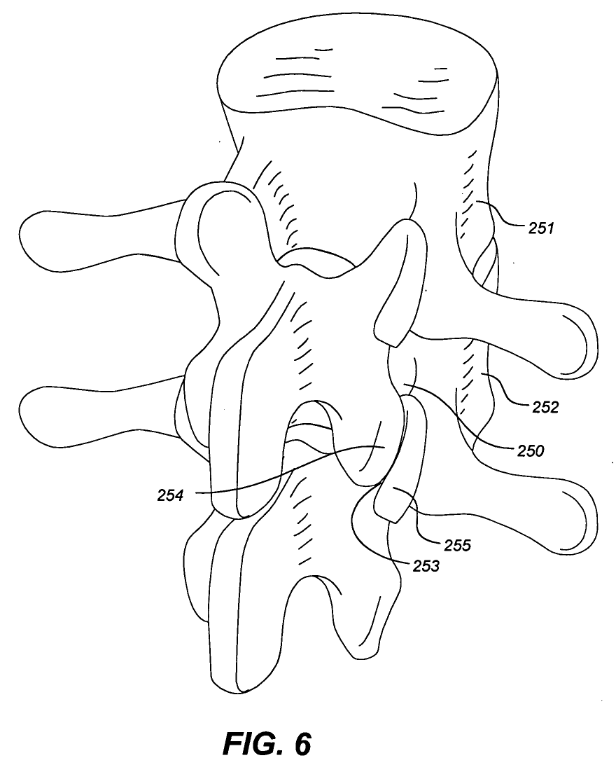

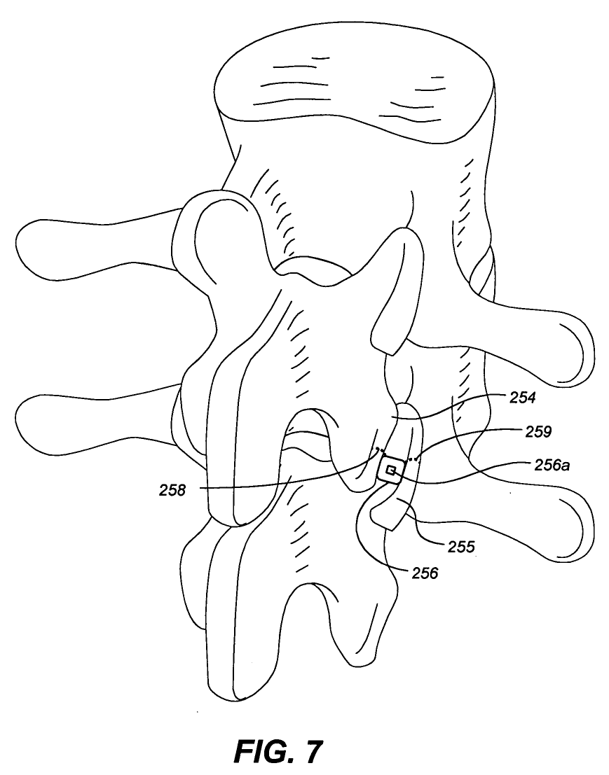

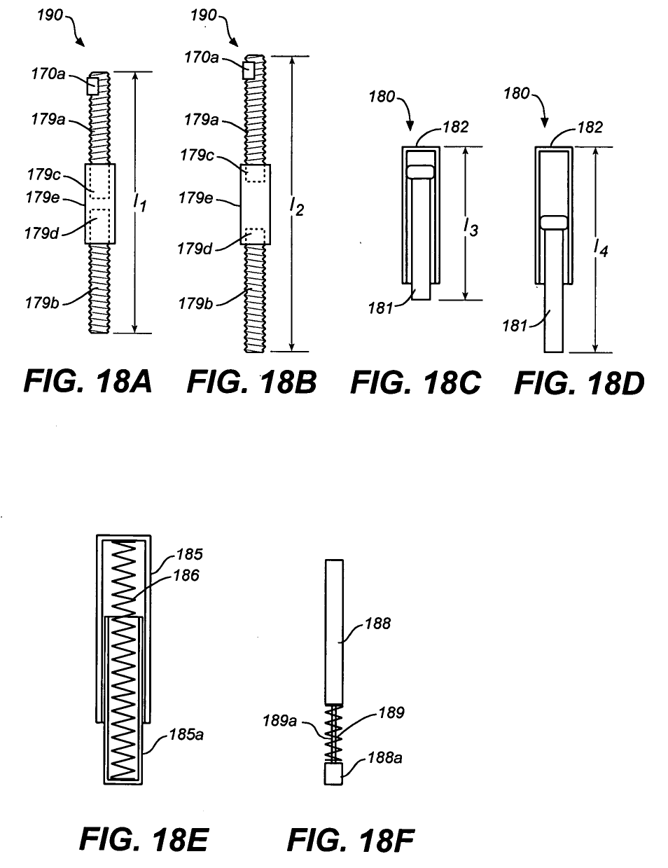

Referring to FIG. 7, the portion of the spine of FIG. 6 is illustrated with a facet distracter implant 256 in place between the cephalic facet 254 and the caudal facet 255. The implant 256 comprises a distracting portion 257 and anchors 258, 259 comprising barbs or bone anchors. The distracting portion may include a distracting element as described with respect to FIGS. 18A-18E herein. The anchor 258 is positioned in bone above the cephalic facet 254 while the anchor 259 is positioned in the bone below the caudal facet 255. The facet distracter implant 256 includes a sensor 256a, the type of which may be selected to sense one of a number of different parameters. Pressure sensors, strain gauges, or other sensors may be used to sense load seen by the facet joint. This information may be used to monitor the condition of the facet joint or determine if fusion may be necessary. The other facet joint implants described herein may also include similar sensors.

The procedure for implanting the device generally includes opening the zygapophyseal joint capsule with a scalpel. Then the adjacent vertebrae are distracted by one of a number of known distraction methods or by distracting the joint mechanically using devices such as a wedge or expanding rod or balloon between adjacent spinous processes, or between other parts of adjacent vertebrae. The tissue between the facets 254, 255 is then debrided and/or denervated. The implant is then inserted between the facets 254, 255 after the joint is distracted. The anchors 258, 259 engage the interfacing portions of the bone of the facets 254, 255.

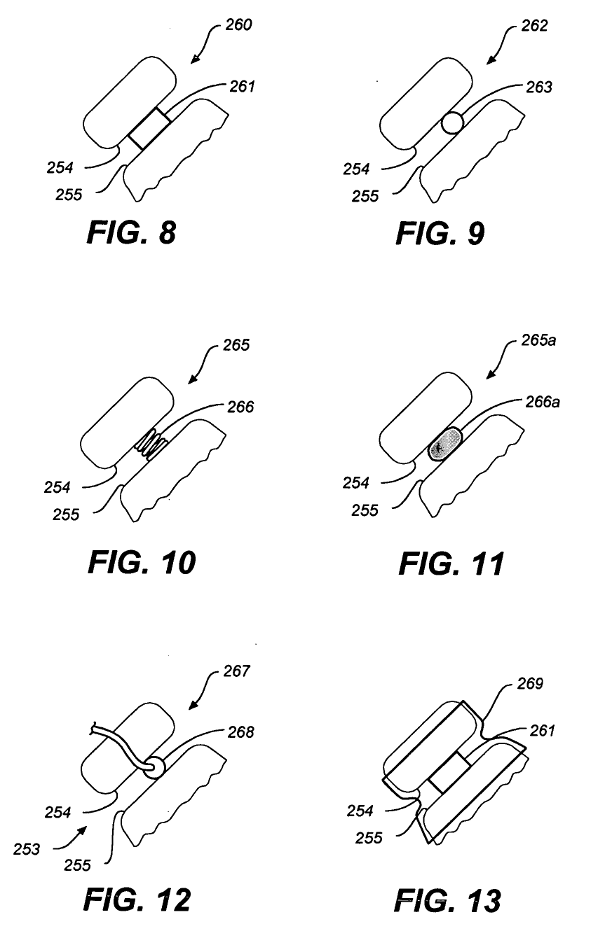

FIG. 8 illustrates a distracter implant 260 positioned between facets 254, 255. The distracter implant 260 comprises a block wedged 261 between the facets 254, 255. In FIG. 9 an alternative distracter 262 implant comprises a ball 263. In FIG. 10 an active distracter implant 265 comprise a coiled spring 266. In FIG. 11, the distracter implant 265a comprises an expandable polymer 266a, e.g., a hydrogel or expandable gel foam. In FIG. 12 the distracter implant 267 comprise an expandable member 268 that may be expanded to distract the joint 253 by inflating with a curable polymer, a liquid, gas or other material. The distraction may occur after implantation to adjust the level of distraction. The expandable member may also be adjusted after implanting by increasing or removing the inflation medium, e.g. using a needle or accessing the member through a one-way valve. FIG. 13 illustrates a shrink-wrap 269 placed partially around the joint 253. The shrink-wrap or other material comprises, e.g., a Dacron material that holds the block 261 or other implant in place between facets 254, 255. The material may encourage ingrowth of tissue. The material may be coated with a material that reduces tissue ingrowth to permit the joint to move or reduces adhesions to prevent pain. The material may include burrs or barbs that secure the material to the bone or it may be secured, e.g. with suture anchors. The implants may be constructed, for example, of a metal, polymer or ceramic, may be coated or imbedded with therapeutic agents (e.g. a steroid or lidocaine) or other material.

Another aspect of the invention is to allow for partial or complete removal of a facet or facet joint in the treatment of spinal stenosis, or for aggressive laminectomy in the treatment of spinal stenosis. A device in accordance with the invention may serve as a shock absorber that allows for partial or complete removal of degenerative facets. Accordingly a device is provided that shares some of the spinal column's axial, torsional, and shear loads, replacing the native painful, deformed, or dysfunctional facet.

In accordance with one aspect of the invention, a distraction system is provided where the system is anchored on opposite sides of a motion segment of a facet joint that would benefit from distraction. On opposite lateral sides of the motion segment, an expandable rod, screw, or other columnar support structure is attached. The length of the support structure may be adjusted to determine the degree or amount of distraction. Additionally, a spring or shock-absorbing element may be included in the distraction device.

FIGS. 14A and 14B illustrate a support prosthesis configured to provide support of the spine where a facet has been removed in whole or in part. The support prosthesis 270 comprises a support rod 279 anchored into a pedicle 273 of a first vertebra 271 through a screw head of a pedicle screw 275. The support rod 279 extends through an opening 278 in the spinous process 277 to a pedicle screw 276 anchored in contralateral pedicle 274 of a second vertebra 272. The support rod 279 is oriented at an oblique angle with respect to the rotational axis of the spine, i.e. at an oblique angle with respect to the median and horizontal planes of the spine, and over the region 279a from which the facet was removed. The support rod 279 may include shock-absorbing properties, for example, as discussed above with reference to FIGS. 18E-18F. The rod 279 at least in part supports the load that was previously borne by the removed facet joint when it was intact. The support rod 279 also provides distraction for the joint. The spinous process 277 may include reinforcement or a support structure such as described herein. The rod 279 may be constructed of a materiel that permits flexing and twisting motions, such as, e.g., a suitable polymer material. The superior part of the rod 279 may alternatively be anchored in the lamina, spinous process or attachments to the posterior elements of the vertebra.

According to another aspect of the invention a rod is provided that is anchored to with pedicle screws with screw heads made of or attached to swivel collars, polyaxial heads, or other movable fasteners to allow for near physiologic levels of motion of the spinal motion segment. Angular movement may be provided where a distracting element attaches on either side of a motion segment so that when distracting or lengthening the device, there is accommodation in the device for the change of angle that occurs.

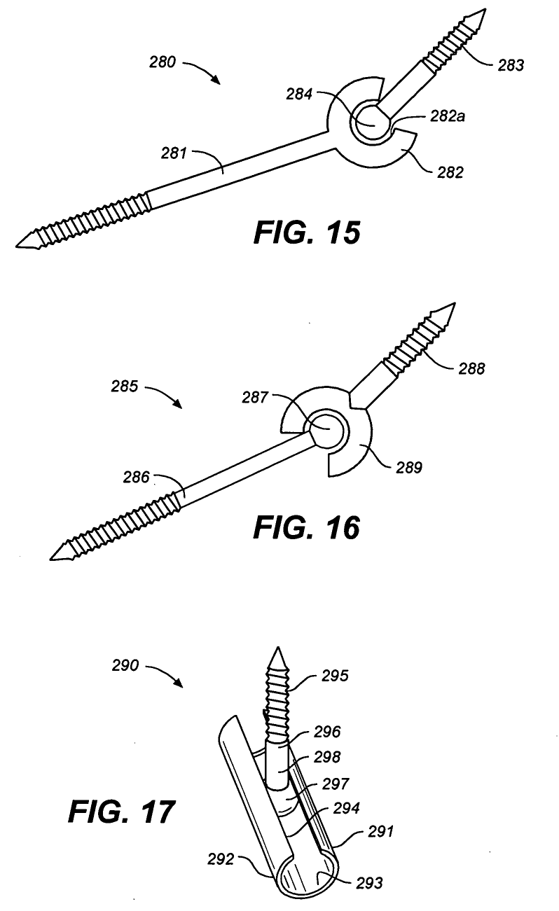

FIG. 15 illustrates an enlarged portion of a spinal prosthesis. The prosthesis 280 may provide support of the load on the spine where a facet has been removed or may provide other support or distraction. The prosthesis 280 comprises a distraction bar 281 used to distract a motion segment of the spine in a number of manners including the distraction devices described herein. A pedicle screw 283 is screwed into a pedicle of the spine or other anatomical location. The distraction bar 281 includes and articulating cup 282 having an inner surface 282a. The pedicle screw 283 has a ball 284 received by and coupled to the cup 282 of the distraction bar 281. In addition to shock absorbing capabilities described in various embodiments herein, the distraction bar 281 also articulates with a portion of the spine to which the pedicle screw 283 is attached.

FIG. 16 illustrates a variation of the prosthesis 280 described with respect to FIG. 15. The prosthesis 285 comprises a distraction bar 286 and an articulating ball 287 configured to engage and couple with an articulation cup 289 of a pedicle screw 288. The prosthesis 285 operates in a similar manner as prosthesis 280.

FIG. 17 illustrates a variation of the prostheses 280, 285 described herein respectively with respect to FIGS. 15 and 16. The prosthesis 290 comprises a distraction bar 291 having an end 292 with a lumen 293 for slidably receiving the end 296 of a pedicle screw 295. The end 296 of the pedicle screw 295 comprises a ball portion 297 attached to a neck 298. The ball portion 297 is configured to slide within the lumen 293 of the distraction bar 291 which contains the ball portion 297. The neck 298 of the pedicle screw 295 extends out of the distraction bar 291 through a longitudinal slit 294 that slidably receives the narrower neck portion 298 of the pedicle screw 295.

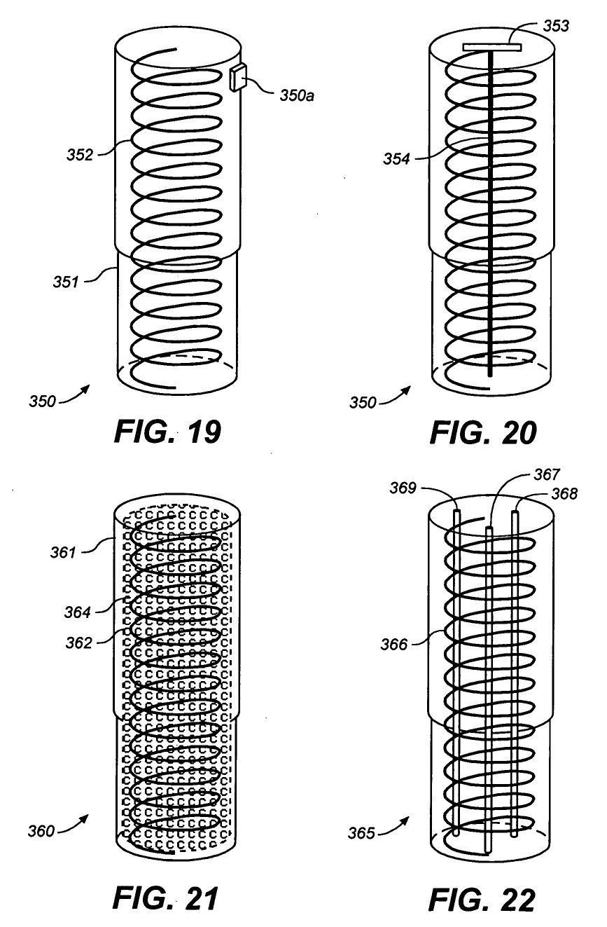

A variety of distraction systems are contemplated for distracting the adjacent vertebrae (including but not limited to the distractions systems disclosed herein), e.g., an expandable screw or rod or plate, telescoping implant, a distraction jack, an inflatable column, a column that lengthens when exposed to heat, fluids, ultrasound, or other biological, physical, or chemical catalysts (using, for example, a device constructed of a shape memory alloy or rheostatic fluids). The amount of distraction may be controlled remotely, by radiofrequency, electromagnetic energy, electrical, heat, ultrasound, and other means. The distracting member for example may comprise a remotely actuated realignment device or solenoid. The distraction can also be adjusted percutaneously or remotely according to one of these variations. The adjustments may be made over time, particularly if the disease progresses or other anatomical changes occur. This would allow adjustment of the amount of distraction as needed to a patient's symptoms long after surgery. The distraction adjustment may also be done with patient feedback. The distraction devices may also include a variety of different types of sensors that sense changing loads on the spine or on the device. For example, the distraction device may include a pressure sensor or a strain gauge. As noted above, the distraction device with spring properties may include a freeze or lock (for example, as described with respect to FIGS. 19-22 herein) that permits the device to be immobilized should a fusion type procedure be necessary to immobilize a patient's spine, for example at a later date with further wear or progression of disease. The flexibility or stiffness of the device may also be incrementally or progressively adjusted as described with respect to FIGS. 19-22 herein.

FIGS. 18A-18F illustrate an enlarged view of distraction elements that may be used as distractors in distraction rods, for example incorporated into the rod as set forth in FIGS. 7 and 14A-17. FIGS. 18A and 18B illustrated an enlarged view of support rod 279 illustrated in FIGS. 14A and 14B. comprises two opposing rods 179a, 179b with abutting ends 179c 179d and an adjusting device 179e connecting the threaded abutting ends 179c, 179d. In FIG. 18A the ends 179c, 179d of the opposing rods are immediately adjacent each other and the length l₁ of the rod is relatively shorter. In FIG. 18B, the extension by the adjusting device 179e has moved the ends 179c 179d apart from each other and the length l₂ of the rod 279 is relatively longer. The rod 279 is operable to be extended and locked into an extended position whereby a joint is distracted. The rod 279 may be extendable after implanted to slowly distract the joint until a desired result (e.g., reduction of patient pain or discomfort) is achieved or degree of release of stress on a joint is achieved. This can be visually determined, determined according to patient feedback or determined by a sensor 170a positioned on or adjacent the implanted distraction system 170. (Here it is near the attachment site to the bone.) The sensor 170a may be a strain gauge, an accelerometer, a piezo-electric film or other sensor that can be used, positioned or configured to determine a mechanical load on the distraction device. The sensor 170a may also be a stand alone sensor positioned in or adjacent a distracted joint and configured to sense a parameter indicative of forces at the joint. The sensor may include an electronic circuit that is configured to telemetrically send a signal containing information correlated to such sensed forces. The electronic circuit may be a passively powered device from an external power source where the external device may interrogate the sensor for information. The electronic circuit may also include signal processing circuits or memory. The rod 279 may include a remotely actuable length adjusting device. For example, the rod 279 may include a mechanical, magnetic or other adjusting device such as a small machine (e.g. a solenoid, a piezoelectric motor or other electromechanical device) that may actuate or move the rod to adjust the degree of distraction. The adjusting device 179e may be actuable by the patient or provider or may automatically adjust, may be adjusted by circuit 179f (that may be telemetrically controlled and/or powered) or may adjust the distraction on demand based at least in part on information sensed by the sensor 170a via control signal through electronic circuit 179f. The rod 279 may also include a mechanism that is designed to break or fail when a certain force is applied to the device. One or ordinary skill in the art may design the device to release, disengage, fail or break with application of a predetermined or selected force by creating a release mechanism or faults in the material or selecting material or structure specifications. For example the device may be constructed to operate under given normal operating forces but to release, disengage, fail or break prior to a force sufficient to fracture the bone.

FIGS. 18C and 18D illustrate an enlarged view of a variation of a distraction element in accordance with the invention that may be used with any of the distraction rods herein. The distraction element 180 comprises opposing rods 181, 182 with rod 181 slidably positioned at least partially within rod 182. The rods 181, 182 longitudinally slide with respect to one another to vary the total length of the distraction element 180. The inner wall of the rod 182 and outer wall of the rod 181 are configured to engage with a detent mechanism, cammed surface or other interference type fit mechanism, when the rods 181, 182 are rotated or actuated or distracted with respect to each other to thereby fix the length of the distraction element 180. FIG. 18C illustrates the distraction element 180 with a relatively shorter length of l₃ and FIG. 18D illustrates the distraction element 180 with a relatively longer length of l₄. The rods 181, 182 may also be simple telescoping tubes that can be crimped or welded or ratcheted together when a desired distraction length is determined.

Referring to FIG. 18E a distraction element 185 that may be used with a distraction device, is illustrated containing a coil or spring-like member 186 where the spring is longitudinally biased so that the coil tends to lengthen, providing a distraction type force. The distraction element 185 may be converted into a rigid or less flexible distraction rod or may be adjusted in flexibility in a manner as described with respect to the devices illustrated in FIGS. 19-22 herein.

Referring to FIG. 18F a distraction element 188 that may be used with a distraction device in accordance with the invention, is illustrated with a spring 189 on one end. The spring 189 is longitudinally biased in a lengthening direction as the spring member 186 described herein with reference to FIG. 18E. The spring 189 is configured to permit movement in a plurality of directions and/or planes. A rubber member 189a is positioned inside the coil and acts to dissipate energy or absorb shock. Thus, the distracting rod 188 provides a distracting force in combination with shock absorbing properties. The rod 188 may also be converted to a rigid distraction rod in a manner described above with reference to the distraction rod 185.

FIGS. 19-22 illustrate convertible or adjustable dynamic stabilization devices for joints. These devices may be used, for example in distraction elements described herein with respect to FIGS. 18A-18E. The stiffness or flexibility of the device may be altered or titrated after implantation to adapt the stiffness to a particular patient, and/or to adjust the stiffness over time, for example when laxity of the joint increases with age. Referring to FIG. 19 illustrates a dynamic stabilization prosthesis 350. The prosthesis comprises a flexible coil 352 contained in a tube member 351 comprising telescoping tubes. The prosthesis 350 may be used in a number of manners affixed across a joint motion segment to dynamically stabilize the joint. The coil 352 may be energy absorbing. The coil 352 may also be configured to exert a distracting force on the joint when implanted. FIG. 20 illustrates the dynamic stabilization prosthesis 350 of FIG. 19 converted to a rigid or more rigid prosthesis. The prosthesis 350 includes a slit 353 for receiving a rigid wire member 354. In FIG. 20 the rigid wire member 354 is inserted into the slit 353 to form the prosthesis from a dynamic prosthesis into a rigid prosthesis. As an alternative to a rigid wire member, a flexible coil of a selected stiffness may be inserted to change the stiffness of the dynamic prosthesis. The tube may alternatively comprise a ferromagnetic material contained therein and an electromagnetic field is applied that causes the prosthesis to become stiffer. The field may be varied to provide a variety of gradients in stiffness. The device may also include a sensor that operates as sensor 170a described herein. Feedback may be provided and the stiffness of the prosthesis adjusted accordingly. The stiffness may be varied when implanted using patient feedback so that the implant is more or less flexible depending upon an individual patient's needs. In addition the stiffness may be changed at different times during the course of the implants lifetime. For example, the stiffness may be increased when an increased amount of stabilization is required.

FIG. 21 illustrates an alternative prosthesis 360 also comprising a flexible coil 362 contained in a tube member 361. The tube member is configured to receive a fluid material such as a curable polymer 364 that cures in the tubular member to create a rigid prosthesis. As illustrated in FIG. 21 a rigid prosthesis is formed from a dynamic prosthesis by injecting the polymer material 364 into the tubular member 361. The flexibility/stiffness properties of the prosthesis may be selected by selecting such properties of the polymer to be injected.

As illustrated in FIG. 22 a flexible prosthesis 365 is illustrated. The flexibility of the prosthesis 365 is adjustable by injecting a polymer material into one or more of the columnar cavities 367, 368, 369. The polymer may be injected into each cavity at a different time so the stiffness of the prosthesis may be increased gradually over time. The stiffness/flexibility properties of the polymer injected may also be selected according to a desired stiffness/flexibility of the implant.

According to an embodiment of the invention, the dynamic stabilizer may comprise a shock absorber that has both energy absorbing and energy dissipating properties. The tension band effect of the posterior columns may also offload the pressures borne by anterior column of the spine. So in addition to helping to protect the facet joints, other aspects of the invention would help slow the progression of degenerative disc disease, annular degradation, disc herniation, and vertebral compression fractures.

Another aspect of the invention is to supplement implants or repair procedures of the anterior column with a posterior shock absorber device (rod, screw, plate). Examples of these implants or procedures include total disc replacements, annular repair, artificial nucleus, and vertebroplasty/kyphoplasty.

Another aspect of the invention is to supplement implants or repair procedures of the posterior column with a shock absorber rod. Examples of these implants or procedures include interspinous distraction wedges, facet joint replacements, and posterior arch replacements.

Another aspect of the invention provides a posterior support implants with shock absorbing properties, to decrease or remove the load experienced by the facets. Implant components may include springs, coils, hydraulic or fluid filled piston chambers, or elastic materials. Each end of the device could be anchored in such a fashion so the rod bridges the facet joint, reducing the loads borne by the joint. This is believed to reduce wear of the facets and resulting pain and altered spinal biomechanics

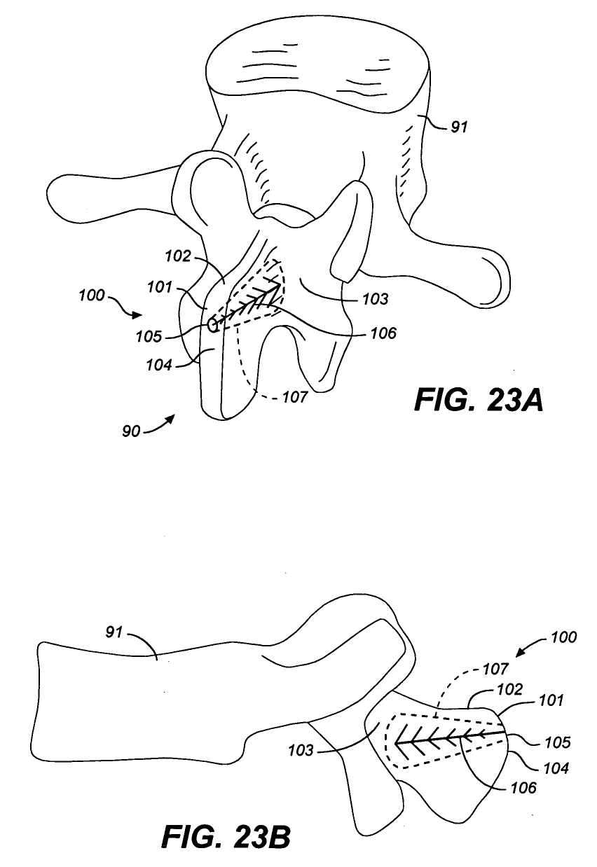

FIGS. 23A and 23B illustrate a partial cutaway view of a reinforced posterior arch 100 of a first vertebra 91 of a spine 90, including a spinous process 101 and lamina 103. The first vertebra 100 of the spine 90 as illustrated includes a first spinous process 101 with a superior portion 102 having a posterior ridge 104 into which a hole 105 is drilled. The hole 105 may be drilled with a drill, a trocar, a large bore IV needle or similar sharp object through the external and relatively hard cortical bone, to reach the internal cancellous bone within the spinous process 101 and adjacent the lamina 103.

Once the cancellous bone is accessed, optionally, a tool such as a balloon tamp, or other expandable member or small crushing or drilling member is used to create a cavity 107 or cavities within the cancellous bone by compressing, crushing or drilling out the bone material. X-rays may be used to determine how far to drill into the bone. The cavity 107 may be in the spinous process, through to the base of the spinous process, or through the spinous process and into the lamina. In one embodiment the cavity is cone shaped or widens as it moves anteriorly towards the lamina.

A reinforcing material is then delivered into the cancellous bone or cavity 107 of the spinous process 101 and/or within the lamina 103. The material is selected to provide reinforcing properties to the spinous process 101 and/or lamina 103 sufficient to support (whether alone or in combination with other support elements) a spine support structure, a prosthesis, or other device attached to the spinous process and or supported lamina. The material may be a bone cement or polymer with strength and hardness properties selected to provide sufficient reinforcement to the region so that the spinous process may be used at least in part, to support an implant structure for attaching to and manipulating the biomechanics of the spine. Examples include but are not limited to polymers such as acrylic cement developed for use in vertebroplasty procedures. The material may be a flowable polymer material that cures within the cavity. Suitable materials may be readily selected by one of ordinary skill in the art.

Reinforcement structures may be placed within the cavity prior to, during or after injection of flowable material for further strength properties. As illustrated, an additional support structure 106 is provided within the cavity. The support structure 106 may be inserted through a cannula and released to expand as a spring-like or self-expanding member, into the cavity. The support structure 106 provides further support of the spinous process and/or lamina. Alternatively, or additionally, one or more posts or struts may be provided within the cavity or extending out of the spinous process or lamina from the area of cancellous bone, to supplement the support of the spinous process or lamina in combination with the polymer or other curable material. The reinforcement structures may be formed of a number of different materials such as, e.g., a metal or biocompatible polymer. Such reinforcement structures may also be used in other bony areas of the spine including the vertebra, the pedicles, facets, the transverse process, etc.

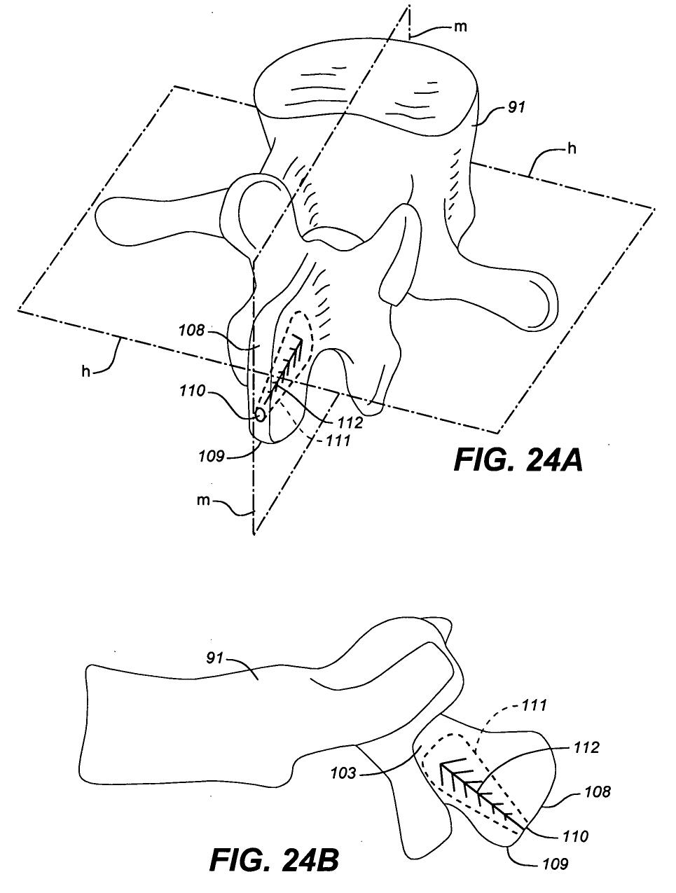

As shown in FIGS. 24A and 24B, an inferior portion 109 of a spinous process 108 may also be reinforced. Similarly a hole 110 is drilled in the inferior portion of the spinous process 108 and a cavity 111 is formed. The cavity 111 is similarly filled with a curable polymer and is reinforced by reinforcing elements 112 positioned within the cavity.

The reinforcement structure may be used in a number of applications including increasing the strength of healthy bone to support the and fixation of orthopedic implants, as well as increasing the strength of bone weakened by osteoporosis, chronic steroid use, avascular necrosis, weakened by injury and cancer involving the bone. According to one aspect, the reinforcement structure comprises a material that provides sufficient strength including but not limited to suitable polymers, e.g. PEAK, titanium, steel and carbon fiber.

The stabilizing and/or distracting devices described herein may be formed of a material that provides sufficient column strength including but not limited to suitable polymers, e.g. PEAK, titanium, steel, and carbon fiber.

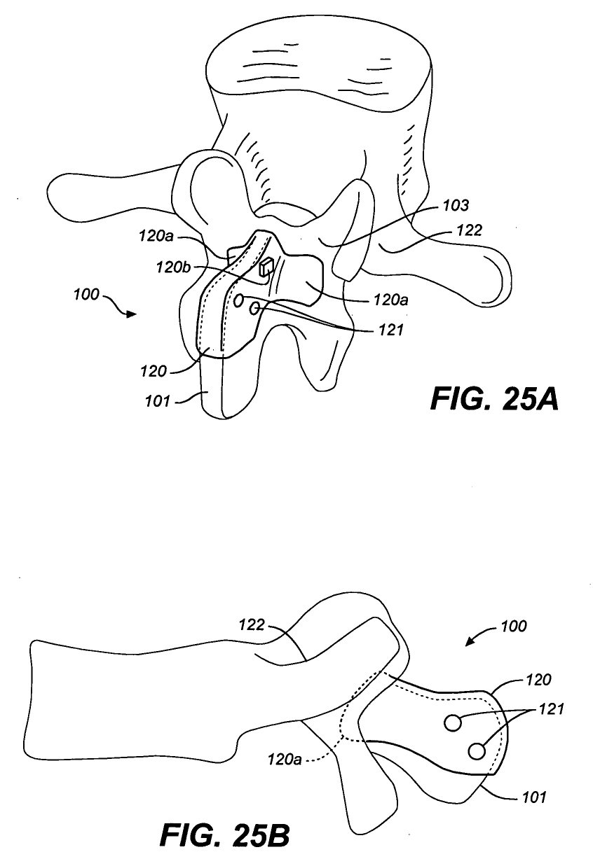

Referring to FIGS. 25A and 25B, an alternative support structure 120 is illustrated. The support structure 120 allows the anchoring of implants under physiologic loads on the spinous process 101 while shielding underlying bone from loads that would normally cause the bone to fracture. (The implants may alternatively or in addition be anchored or attached to the lamina 103, e.g., with addition of small screws, barbs or adhesive that engage with the lamina while avoiding injuring the spinal cord surrounded by the lamina.) The support structure 120 comprises a hood like element positioned over the posterior arch 100, i.e., the spinous process 101 and lamina 103 of a spine 90. The support structure 120 may be made of a moldable or malleable material (e.g. putty, formable ceramic, clay-like material, or a moldable polymer or malleable alloy or metal) that cures into or forms a solid, strong structure. Heat, light, catalysts, precursors, or local pressure and force, for example, may be used to make the hood moldable or firm. The support structure of filling material to support the spinous process may be constructed or formed of moldable composites that can cure into hard material such as, e.g., ground glass powder or glass fiber fillers mixed into an acrylic matrix and activated with light or other biophysical modalities. Other cements or other curable materials may be suitable as well. The support structure 120 further comprises openings 121 to guide drill bits and/or for the placement of screws, reinforcement posts, or other instruments or supplemental support structures. The support structure 120 may be anchored on the posterior arch by mold bending or forming the structure about the anatomy. The support structure 120 may be anchored into the lamina or spinous process by anchoring elements, such as, e.g., screws or barbs. The support structure 120 may also be anchored via screws or posts. Alternatively, the support structure 120 could be a preformed implant with contours that fit the anatomy of the posterior arch 100 or that are malleable or moldable to the anatomy. Also, the support structure 20 may be anchored into the pedicles 122 with screws, into the underlying bone with barbs, screws, bone anchors, or adhesives, over the edges of structures with hooks, or may be constructed of a plurality of pieces that may be assembled into one piece around the bone. Wings 120a of support structure may be placed over the lamina to spread the force of any device attached to the support structure 120.

As illustrated in FIGS. 25A and 25B, a sensor 120b is positioned on the support structure 120. The sensor 120b may be embedded in the material. The sensor may sense stress on the support structure 120 from implants secured to it, or may sense other information that may be desirable to monitor. The sensor may include a communication element configured to communicate sensed information to an external device, e.g., when interrogated.

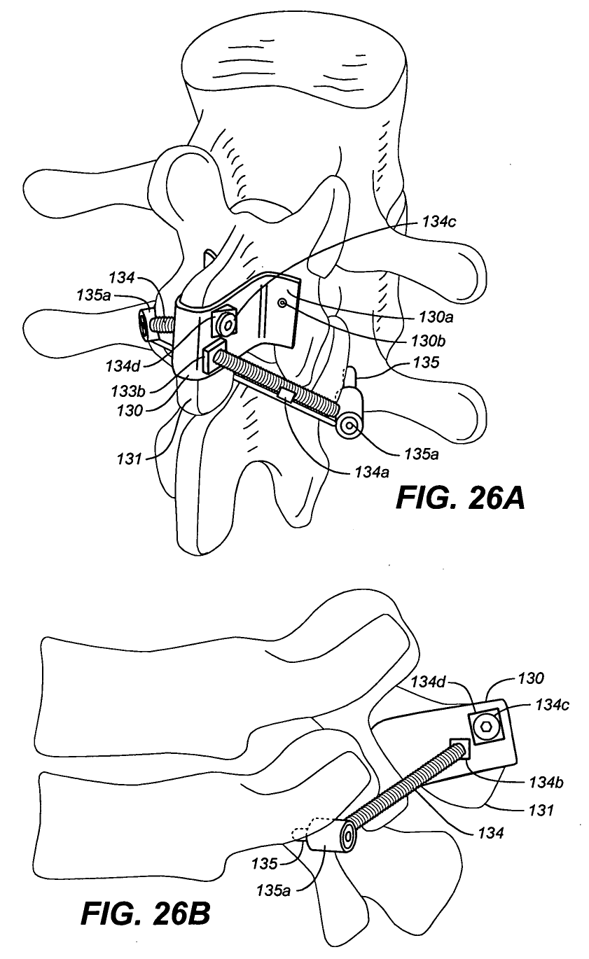

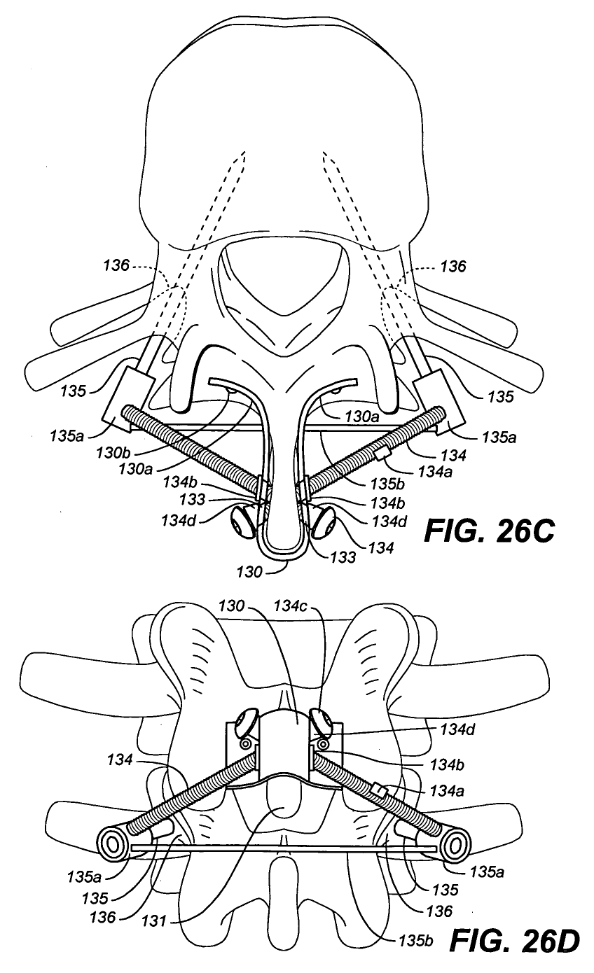

Referring to FIGS. 26A-26D, a support structure 130 is illustrated positioned over a posterior portion 132 of a spinous process 131 with wings 130a over the lamina 103 including small screws 130b into lamina 103. Wings 130a may help spread the force from any devices attached or coupled to the support structure 130. Pedicle screws 135 are anchored into pedicles 136 and are further anchored into the spinous process 131 through screws 134 positioned through holes 133 in the support structure 130. As shown in FIG. 26C, the screw 134 includes a sensor 134a that may be used to sense loads on the device. Use of such sensors is described further herein. The pedicle screw 135 includes a screw capture device 135a for receiving a screw or rod of a spinous process screw or other rod. The capture device 135a may be a polyaxial head of a pedicle screw it may include a hole, a threaded screw hole with a washer or cap. Cross bar 135b is positioned across the spine between heads of pedicle screws 135 to prevent pedical screws from creeping laterally. A wedge shaped nut 134d between the head 134c of the screw 134 and the support structure. Another nut 134b may be positioned between support structure 120 and pedicle screw, and secure against the support structure 120. These features may be used in a similar manner in the embodiments described herein.

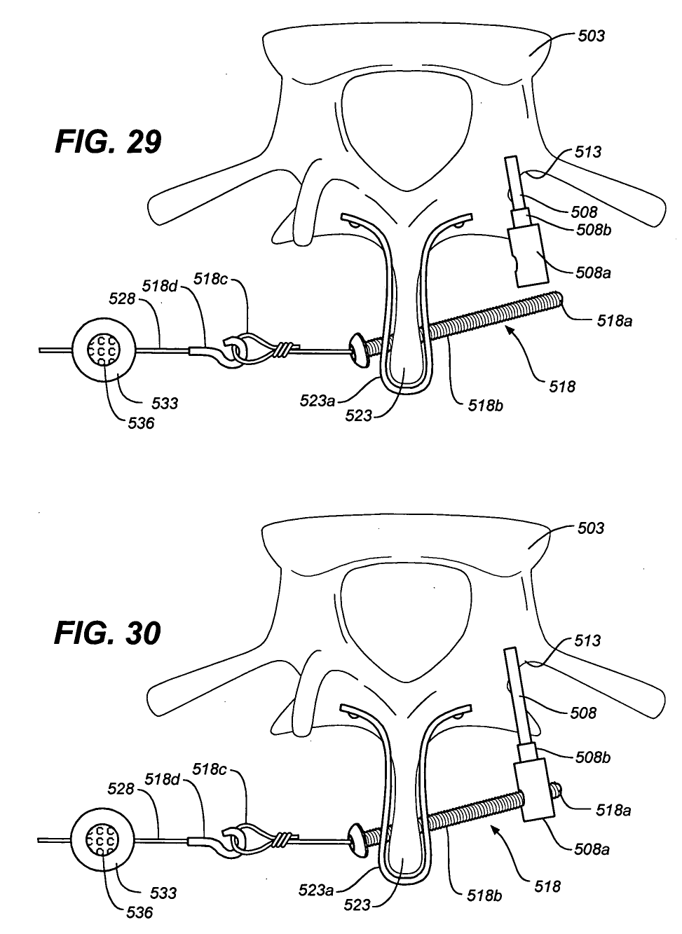

The pedicle screw 135 may be configured to telescope outwards or inwards to be positioned to receive the screw head or rod of a spine device as shown in FIGS. 29 and 30. Referring to FIGS. 29 and 30, a pedicle screw 508 is configured to telescope outwards or inwards to be positioned to receive the screw head or rod of a spinous process screw 518. The spinous process screw 518 is shown in FIG. 29 where, given the trajectory of the spinous process screw 518, its end does not intercept the capture device 508a of the pedicle screw 508. As shown in FIG. 30 the pedicle screw's trunk 508b is lengthened with a telescoping or other similar lengthening mechanism so that the end of the spinous process screw 518 may be positioned in the capture device 508a.

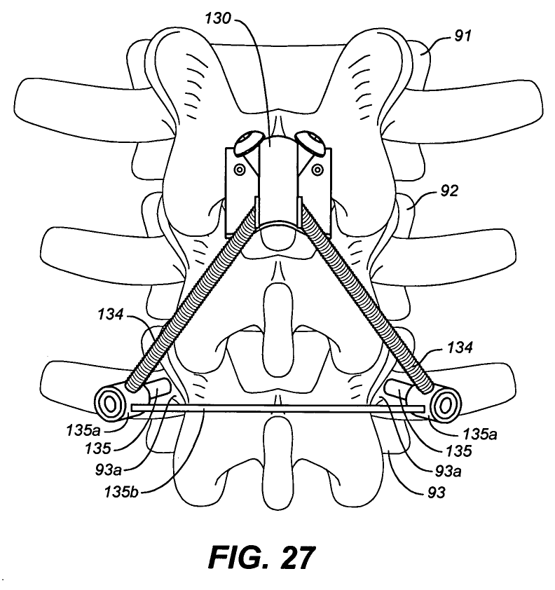

FIG. 27 illustrates the spinous process screws 134 coupled to a spinous process 101 of a first vertebra 91 through a hood or support structure 130 in a manner similar to that described above with respect to FIGS. 26A-26D. The screws 134 extend bilaterally across the posterior of a second vertebra 92 and are anchored to capture elements 135a of pedicle screws 135 anchored into pedicles 93a of a third vertebra 93.

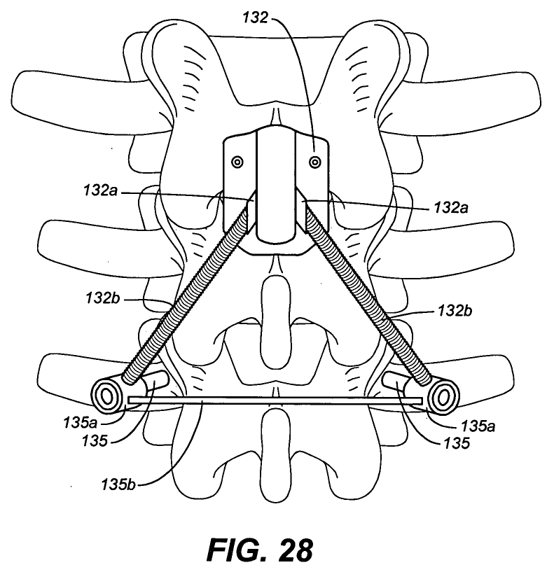

FIG. 28 illustrates a device for stabilizing or distracting the spine with pedicle screws 135 and cross bar 135b positioned as in FIG. 26D. Hood structure 132 includes openings for receiving screws 132b coupled to the hood 132 on one end and to the heads 135a of pedicle screws 135 and on the other end. The screws 132b do not penetrate the spinous process. Obliquely threaded nuts secure the screws 132b against the hood 132.

The reinforcement or supporting devices described herein may be used in conjunction with a number of different spine devices, including, for example, the various distraction, fusing or dynamic stabilizing devices described herein. The hoods or reinforcement devices herein may also be customized, for example by using stereolithography. The hoods or reinforcement devices may be used for example with a brace.

The devices described herein may be coupled to the spinous process using minimally invasive techniques. These techniques may include percutaneously accessing the spinous process and/or using dilators to access the spinous process at an oblique angle with respect to median plane m and/or horizontal plane h through the spine of the patient. An oblique skin stab wound is made to navigate to the spinous process, which may be exposed under direct vision. The spinous process screw or other distraction device is then screwed or positioned through the spinous process across or through the facet joint, and into a pedicle screw or attachment device stabilizing the facet joint. A similar screw may also be placed from the spinous process to the contralateral pedicle. The spinous process may be reinforced prior to or after placing the screw or other distraction device.

The various embodiments of the invention described herein may include sensors integrated with or provided on a structural spinal implant. A number of factors may be detected as described herein. Additional factors may include, e.g., local inflammation, pressure, tension, edema, motion, water content, and electrolytes or other chemicals. The sensors allow a doctor to monitor patients for response to healing, or may be used by the doctor to guide serial adjustments to the patient's treatment. For example, measurements from the sensing means could lead the doctor to change the length or tension of a distraction rod or stabilization device. Patients could adjust therapy based on measurements from the sensing device, or could be alerted to notify their doctor should certain measurements be of concern. The sensor is configured to be adjustable to sensed stresses. The sensor may for example, be a strain gauge, a pressure sensor accelerometer, position sensor, imaging device, etc. The sensor may be used in the initial adjustment of the prosthesis or may be monitored over time. The sensor may sense shear/torsion tension/compression. Sensors may sense stresses at various motion segments. The sensor may be used to compare stresses at various motion segments or locations. Various sensors may be selected from sensors that are known to one of skill in the art or that are commercially available.

One embodiment of the invention comprises an anchor device with a therapeutic substance or drug delivery device, e.g. a drug port and/or reservoir, or matrix attached to a vertebra. In one embodiment, the device is anchored adjacent a site near where pain is present. The port is configured to deliver steroids or anesthetic agents via a catheter to a desired location, for example, the facet joint, neural foramen, vertebral body, annulus, nucleus, back muscles, back ligaments, bone metastases, intrathecal space, epidural space, or other targets in, on, or around the spine. The catheter can direct the drug to the correct location by positioning the end of the catheter at a target location. The port is configured to be refilled periodically percutaneously, e.g. using an imaging device and a percutaneously placed needle that can inject the refill into the port, e.g. through a biocompatible polymer or rubber type port access mechanism. The device further comprises a patient actuation mechanism for patient control of drug delivery as needed for pain relief, manually or remotely using a telemetrically triggered delivery from an external telemetry control device. According one aspect of the invention such a device is attached to a boney structure of the spine. Other device that may be attached to the spine may include sensory or therapeutic devices, including nerve stimulators, bone growth stimulators and radioactive seeds.

In addition, a structural implant could be anchored to bone, to which a sensory or therapeutic device could be attached. The sensory or therapeutic device could be placed external to the bone, on the surface of the bone, or internal to the bone.

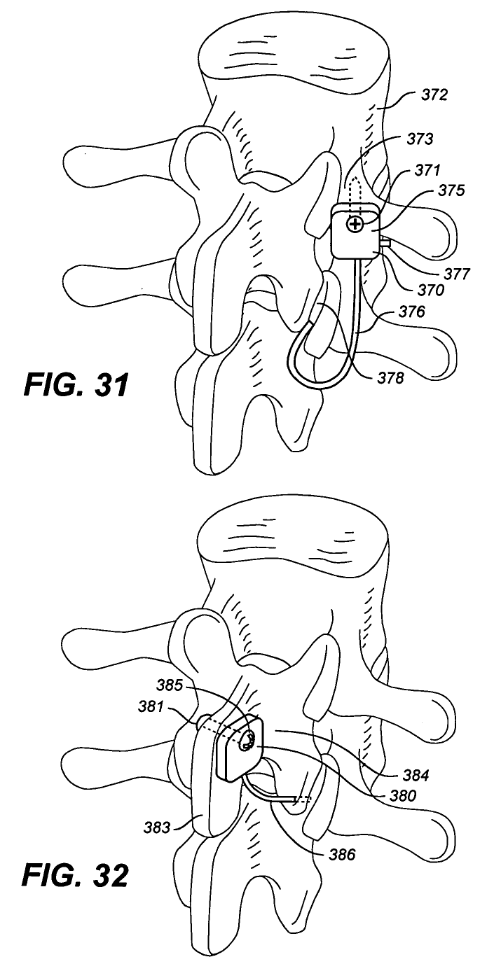

FIGS. 31 and 32 illustrates drug delivery devices 370, 380, respectively, in accordance with the invention. The drug delivery device 370 includes a reservoir 375 attached by an anchor 371 configured to anchor the reservoir 375 to the bone of the spine. In particular, in this embodiment, the anchor 371 comprises a pedicle screw that anchors the device to the pedicle 373 of a vertebra 372. The reservoir 375 includes a catheter 376 in communication with the contents of the reservoir 375 and having an end positioned adjacent or in a zygapophyseal joint 378 where the drug is directed to have a therapeutic effect on the joint 378. The device may include a telemetrically actuable pump mechanism for delivering the drug to the joint upon telemetric actuation by an external control device. The device 370 further comprises a port 377 for receiving (e.g. via a percutaneously introduced needle) into the reservoir 375, refills of the therapeutic substance or drug. Device 380 comprises a similar catheter 386, and reservoir 385 attached by an anchor 381 to the spinous process 383 or alternatively an adjacent lamina 384. The spinous process 383 or lamina 384 may be reinforced prior to attachment of the anchor 381 or may be attached to a reinforcement device positioned at the posterior arch of the spine, as described herein with reference to FIGS. 23A-26D.

1. A spine implant comprising:
a facet prosthesis wherein the facet prosthesis comprises an insert configured to be positioned within a joint capsule between facets of a zygapophyseal joint, wherein the insert comprises:
a member having a first facet interfacing portion and a second facet interfacing portion opposing the first facet interfacing portion.

2. The spine implant of claim 1 wherein the member comprises an expandable member.

3. The spine implant of claim 1 wherein the first and second interfacing portions are configured to permit articulation of the facets.

4. The spine implant of claim 1 wherein the first and second interfacing portions each comprise a curved portion.

5. The spine implant of claim 4 wherein the facet prosthesis comprises a ball-like member.

6. The spine implant of claim 1 wherein the facet prosthesis is configured to exert a distraction force between facets of a facet joint.

7. The spine implant of claim 1 wherein the facet prosthesis includes a flexible portion configured to permit limited motion of the facet joint.

8. The spine implant of claim 7 wherein the flexible portion comprises a flexible polymer forming said member

9. The spine implant of claim 7 wherein the flexible portion comprises an elongate portion extending through said member in said joint and anchored to at least one of said facets.

10. The spine implant of claim 7 wherein the flexible portion comprises an elongate portion extending from said member in said joint and anchored to at least one of said facets.

11. The spine implant of claim 1 further comprising:
a securing member comprising an elongate portion configured to extend through at least a portion of the facet prosthesis; and
an anchor coupled to the elongate member, and configured to anchor the securing member to at least one facet.

12. The spine implant of claim 11 wherein the securing member includes a flexible portion configured to permit limited motion of one or more of the facets of the facet joint.

13. The spine implant of claim 11 wherein the securing member comprises a distracting element.

14. The spine implant of claim 1 further comprising:
a securing member comprising a wrap configured to wrap around at least a portion of the facet joint.

15. A spine implant comprising
a facet prosthesis configured to exert a distraction force between facets of a facet joint wherein the prosthesis comprises:
a curable material injected into the facet joint.

16. The spine implant of claim 15 further comprising
a securing member comprising an anchor configured to anchor to at least one facet of the facet joint and an elongate member coupled to the anchor and configured to extend through the curable material.

17. The spine implant of claim 15 wherein the securing member comprises a dynamic portion.

18. The spine implant of claim 17 wherein the dynamic portion comprises a flexible member.

19. The spine implant of claim 17 wherein the dynamic portion comprises a spring.

20. The spine implant of claim 17 wherein the dynamic portion comprises a shock absorber.

21. A spine implant comprising
a facet prosthesis configured to exert a distraction force between facets of a facet joint wherein the prosthesis comprises:
a first magnet coupled to a first facet and a second magnet coupled to a second facet, wherein the first magnet and second magnet are oriented with like poles facing each other so as to provide a distracting force away from each other.

22. A spine implant comprising:
a facet prosthesis comprising:
an insert configured to be positioned within the joint capsule;
a securing member comprising an elongate portion configured to extend through at least a portion of at least one facet of a facet joint; and an anchor configured to anchor the securing member to at least one facet of the facet joint.
